(19) United States
(12) Patent Application Publication
(10) Pub. No.: US 2010/0006738 A1
(43) Pub. Date: Jan. 14, 2010

(54) Guide wire and catheter management device

(76) Inventor: Paul S. Teirstein, LaJolla, CA (US)

Correspondence Address: GERALD W. SPINKS, 103 EDWARDS STREET, ABBEVILLE, LA, 70510, US

(21) Appl. No.: 12/217,852

(22) Filed: Jul. 8, 2008

(51) Int. Cl. A61M 25/02

(52) U.S. Cl. 248/633; 604/523; 248/68.1

(57) ABSTRACT

A pad designed to be adhered or clamped in place on a surgical field, with pairs of grooves or clamps thereon to hold pairs of guide wires and their associated catheters in place relative to each other. The grooves or clamps can be arranged in a fanned fashion to fan out the free ends of the wire/catheter pairs from other pairs, to keep them from tangling with each other. The grooves or clamps can be adapted to hold the members against longitudinal movement, or to allow longitudinal movement. Labels can be provided on the pad to identify each wire/catheter pair.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

This invention is in the field of equipment used for intravascular medical procedures, specifically flexible elongated members such as guide wires and catheters that are introduced into a patient's blood vessel.

2. Background Art

Invasive vascular procedures like balloon angioplasty and stent implantation require insertion of a guide catheter into the vasculature, usually in the femoral (leg) artery and directing the catheter to the vasculature in need of treatment, such as the heart. Through this catheter, a thin (for example 0.014 inch) wire called a guide wire, is introduced and threaded down the artery to be treated. An additional catheter or other flexible elongated member can be introduced over, or alongside, the guide wire.

At times, the operator must treat or protect more than one vessel using the same guide catheter. In this circumstance, the operator passes two or more pairs of flexible elongated members through the same Y adaptor. The multiple flexible elongated members travel down the same guide catheter and then enter the vessel requiring treatment, with each guide wire and its associated catheter usually entering a different vessel or branch vessel in need of treatment.

The multiple guide wires and their respective catheters enter the guide catheter through the sealable entry site of the Y adaptor. Since the guide wire/catheter pairs have the same point of entry at the Y adaptor, the operator must take steps to keep the wire/catheter pairs separate from each other, and to keep each guide wire identified with the correct catheter. It is important to keep the wire/catheter pairs separate for several reasons. If the wire/catheter pairs become twisted, they will interact with one another; for instance, when the operator moves one wire or catheter, another wire or catheter may also move. Further, different devices, such as stents, are typically passed over the guide wires on the catheters; therefore, if the wire/catheter pairs become twisted with each other, accurate advancement of the associated devices is hindered. Also, since different devices are passed over the different wires on the catheters, the operator must take steps to identify each wire so as not to confuse which wire is going down which vessel or branch vessel.

Currently, the prevalent method of separating wire/catheter pairs is to use layers of sterile towels. However, towels are bulky and difficult to control. Towels securing guide wires also lie on the operative field and if the Y adaptor is moved, the towels tend to stay in place, so that the guide wires may be inadvertently pulled out of the vessel.

A procedure can often involve the use of two wire/catheter pairs, or sometimes even three or four. Typically, the physician takes up a guide wire and its associated catheter and works with them, then puts them down and takes up a different wire/catheter pair and works with it. In the process, the free ends of the wire/catheter pairs outside the Y adaptor can become wrapped around each other. During a complicated procedure, the free ends of the wire/catheter pairs can become greatly interwoven. The cause of this problem is that the various wire/catheter pairs exit through the same port in the Y adaptor, and that their free ends are more or less free to lie along somewhat parallel paths on the surgical field, hindering adequate identification, control, or organization.

It is an object of the present invention to provide an apparatus which will allow the operator to efficiently identify, organize, and manage two or more pairs of guide wires and their associated catheters.

### BRIEF SUMMARY OF THE INVENTION

The present invention provides a small, firm but pliable pad that can lie on the surgical field, separated from the Y adaptor. The pad can be attached to drapes or some other item in the surgical field, such as by clamping, so as to maintain its desired position relative to the Y adaptor. The pad typically has between two and four pairs of grooves or clamps on it, adapted to hold in place two to four associated pairs of flexible elongated members, with each associated pair of flexible elongated members typically consisting of a guide wire and a catheter. The flexible elongated members in each pair are said herein to be “associated” with each other because the guide wire and the catheter are used together. The grooves or clamps can be arranged in a curved layout, so as to “fan out” the wire/catheter pairs and assist in keeping the free ends of each wire/catheter pair separated from the free ends of other pairs. The grooves or clamps are designed to allow easy insertion and removal of the flexible elongated members. Some of the grooves or clamps can be designed to securely hold a wire or catheter against axial movement, while others can be designed to simply hold a wire or catheter in place relative to the other pairs, without restricting the axial movement of the wire or catheter being held.

The pad can have an adhesive surface on its lower side, to allow it to be adhered to a drape or other item to keep it in place on the surgical field, with a selected separation from, and orientation relative to, the Y adaptor. The adhesive surface can be selectively exposed by removing a peel-off cover. Alternatively, tabs can be provided on the pad, allowing it to be clamped in the desired location and orientation by surgical clamps, or other clamping devices.

The pad can also have labels for identifying each wire and each catheter, or each wire/catheter pair. These can be stick-on type labels, or surfaces adapted for writing upon, or they can be pre-molded labeling areas on the pad, with punch-out circles identifying the selected location of each wire/catheter pair.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a plan view of a first embodiment of a guide wire/catheter management device according to the present invention, utilizing grooves to hold the flexible elongated members;

FIG. 2 is a partial section view, showing one type of groove that may be used in the embodiment shown in FIG. 1;

FIG. 3 is a plan view of a second embodiment of a guide wire/catheter management device according to the present invention, utilizing spring clamps to hold the flexible elongated members; and

FIG. 4 is an edge view of the embodiment shown in FIG. 3.

### DETAILED DESCRIPTION OF THE INVENTION

As shown in FIG. 1, a first embodiment of the device 10 of the present invention includes a pad 12 with a plurality of pairs of grooves 14, 15 in its upper surface. Alternatively, the grooves 14, 15 could be formed in a body of flexible material that is mounted on the pad 12. The pad 12 is constructed of a firm but flexible material. Two or more flat tabs 13 can be formed on the pad 12, to provide surfaces to which surgical clamps can be attached, to hold the pad 12 on a surgical drape, in a selected position and orientation relative to a Y adaptor. Each pair of grooves 14, 15 has an associated label 16, identifying the guide wire GW and the catheter CA that are secured in the respective groove pair. A first pair of flexible elongated members FEM1 can be secured in a first pair of grooves 14, 15, near one end of the pad 12, with a guide wire GW in a first groove 14 and a catheter CA in a second groove 15. Similarly, a second pair of flexible elongated members FEM2 can be secured in a second pair of grooves 14, 15 near the opposite end of the pad 12. Also, if desired, a third pair of flexible elongated members FEM3 can be secured in a third pair of grooves 14, 15 near the center of the pad 12. Further, four or more pairs of grooves 14, 15 can be provided on the pad 12, without departing from the present invention.

The guide wire grooves 14 are preferably slits in the upper surface of the pad 12. The slit can be pushed open and the guide wire GW inserted laterally into the slit, which will then close back around the guide wire GW and hold it in place. That is, this type of groove 14 is designed to grip the guide wire GW and prevent it from moving either in the transverse direction TD or in the axial direction AD, with respect to the groove 14, or relative to the pad 12. This is often preferable with guide wires, since they must be prevented from moving relative to the blood vessel in which they have been placed.

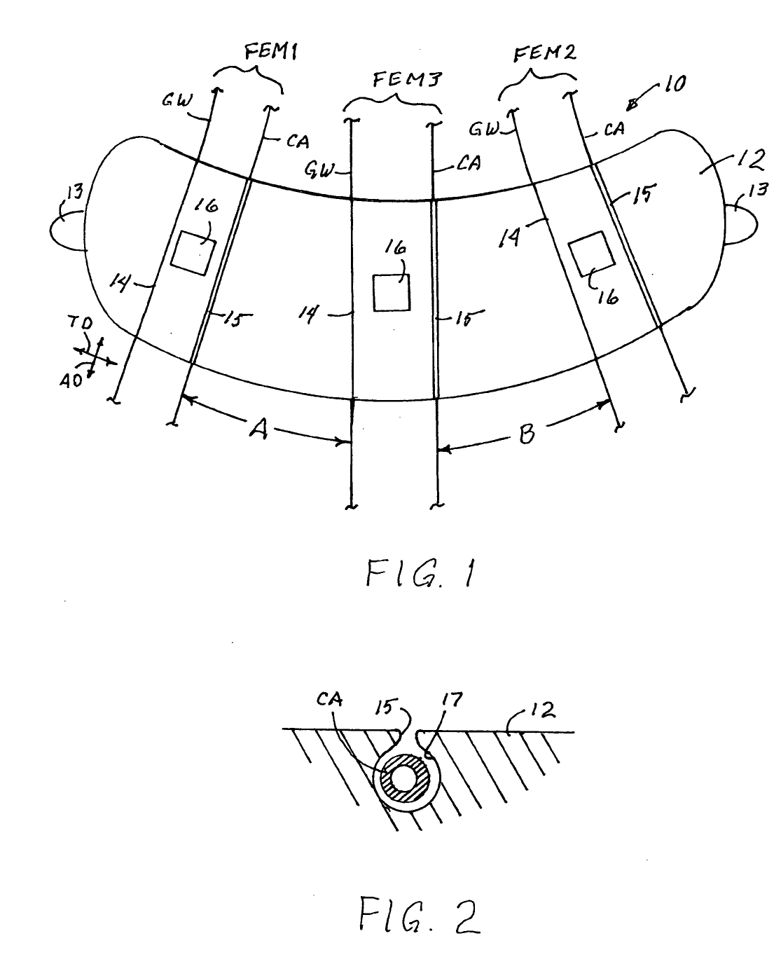

The catheters can also be captured in this slit type of groove, if desired. However, many catheters, being larger in diameter than the guide wires, and carrying larger diameter devices, can be placed in the type of groove 15 illustrated in FIG. 2. That is, rather than a simple slit in the pad 12, this type of groove 15 comprises an open groove, with a tubular cross section 17 below the surface of the pad 12. The surface opening of the groove 15 can be forced open to allow lateral insertion of the catheter CA. Then, the surface opening of the groove 15 will close back around the catheter CA sufficiently to hold it in place against transverse motion relative to the groove 15, or relative to the pad 12. The diameter of the tubular cross section 17 can be selected to closely grip the diameter of the catheter CA, or it can be larger. If the diameter of the tubular cross section 17 fits closely to the catheter CA, it can secure the catheter CA against axial movement relative to the groove 15, and relative to the pad 12. Conversely, if the diameter of the tubular cross section 17 is larger than the diameter of the catheter CA, it can allow the catheter CA to move axially within the groove 15. This can be desirable with some balloon catheters.

The labels 16 can be simply surfaces adapted to be written upon, or stick-on labels with pre-printed identifiers. Or, the labels 16 can be integrally molded into the pad 12, with several possible identifiers listed thereon. These identifiers could include typical guide wire locations, such as LAD, RCA, or CIRC, or even M for Main Artery or B for Branch Artery, etc. Next to each item listed on the label could be a punch-out depression allowing the operator to simply punch a hole next to the item which correctly identifies the location of the associated pair of guide wire and catheter, as the pair is placed in the grooves 14, 15. Alternatively, the operator could simply place a check mark on the label next to the appropriate identifier.

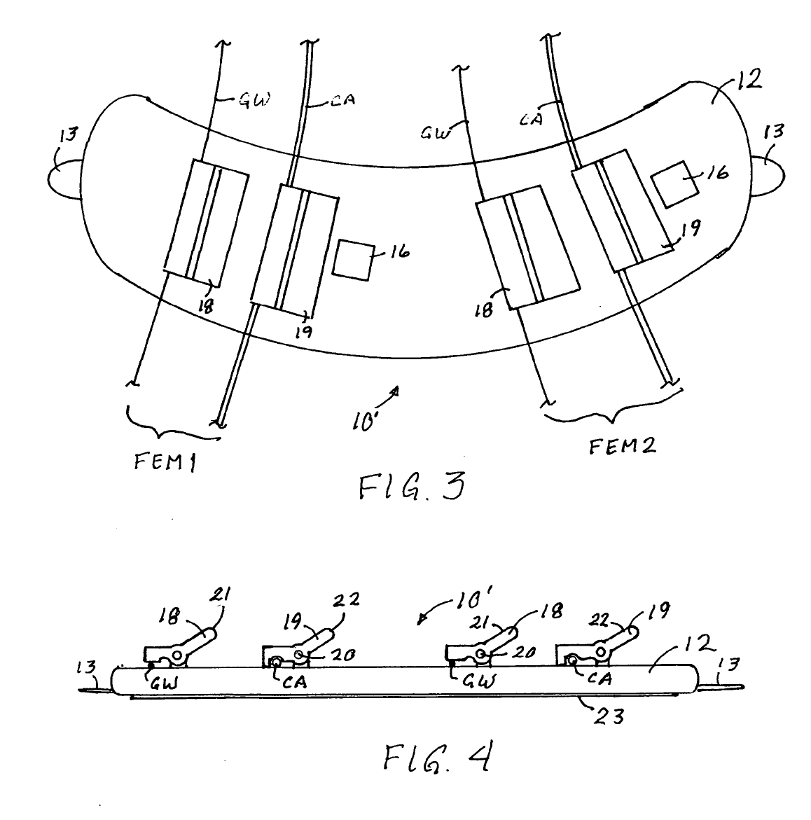

FIG. 3 shows a second embodiment 10′ of the present invention. Rather than grooves in the pad 12, this embodiment utilizes spring clamps 18, 19 to hold the pairs of flexible elongated members in place. That is, the guide wire GW of a first pair FEM1 can be placed in a spring clamp 18 near one end of the pad 12, and the associated catheter CA can be placed in the associated spring clamp 19. Here as before, labels 16 are provided to identify the pair of flexible elongated members that are secured in each pair of clamps 18, 19. FIG. 4 is an edge view of the pad 12 shown in FIG. 3, in a “flattened” view to better illustrate the functioning of the spring clamps 18, 19. A first type of spring clamp 18 could be used to clamp either a guide wire GW or a catheter CA to the pad 12, by simply pressing it against the pad 12. The operator presses down on the wing 21 of the spring clamp 18 to open it, inserts the guide wire GW or catheter CA laterally under the edge of the clamp 18, and releases the wing 21, allowing the clamp 18 to clamp the flexible elongated member tightly against the pad 12. This prevents either transverse or axial movement of the guide wire or catheter relative to the pad 12.

However, another type of spring clamp 19 could also be used where axial movement of the flexible elongated member is desired, such as with some balloon catheters. In this type of clamp, a partial tubular cross-section is provided, within which the flexible elongated member can be placed. As the wing 22 of the clamp 19 is released by the operator, the end of the clamp closes tightly against the pad 12, capturing the catheter CA within the tubular cross-section. If the diameter of the tubular cross-section is larger than the diameter of the catheter CA, the catheter CA is held in place against transverse movement relative to the pad 12, but it is allowed to move axially relative to the pad 12.

The pad shown in FIG. 3 can also have the clamping tabs 13 shown on the first embodiment. Alternatively, or in addition, either embodiment can have an adhesive strip 23 on the bottom of the pad 12, as shown in FIG. 4. The adhesive strip 23 can be covered by a peel-off cover until used. Either method of holding the pad 12 in place is capable of positioning the pad 12 at a desired distance from the Y adaptor, and holding the pad 12 in a desired orientation relative to the Y adaptor.

With either embodiment, it can be seen that the pairs of grooves 14, 15 or the pairs of clamps 18, 19 are arranged in a “fanned” pattern or arrangement on the pad 12, so as to angularly separate or disperse the free ends of the pairs of flexible elongated members FEM1, FEM2, FEM3 from each other. This angular dispersement is illustrated by the angles A, B between the free ends of adjacent pairs of flexible elongated members in FIG. 1.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

I claim:

1. A device for managing associated pairs of flexible elongated members on a surgical field, said device comprising:
a pad;
means for holding said pad in a selected position and orientation on a surgical field; and
a plurality of pairs of attachment devices on said pad, each said pair of attachment devices being adapted to retain a pair of flexible elongated members in a selected position on said pad relative to at least one other pair of flexible elongated members;
wherein said pairs of attachment devices are arranged in a pattern adapted to angularly disperse the free ends of each said pair of said flexible elongated members away from the free ends of other said pairs of flexible elongated members.

2. The device recited in claim 1, wherein said pad is made of a firm but flexible material.

3. The device recited in claim 1, wherein said means for holding said pad comprise an adhesive surface on said pad.

4. The device recited in claim 1, wherein said means for holding said pad comprise at least two tabs on said pad, said tabs being adapted for attaching clamps to said pad.

5. The device recited in claim 1, wherein each said pair of attachment devices comprises at least one spring clamp.

6. The device recited in claim 5, wherein said at least one spring clamp is mounted on said pad.

7. The device recited in claim 1, wherein each said pair of attachment devices comprises at least one groove in a body of flexible material on said pad.

8. The device recited in claim 7, wherein said body of flexible material is integral to said pad.

9. The device recited in claim 7, wherein at least one said groove in each said pair of attachment devices comprises a narrow slit, said slit being adapted to be forced apart to allow introduction of a flexible elongated member into said slit, said slit being adapted to close around said flexible elongated member with sufficient friction to hold said flexible elongated member against axial movement within said slit.

10. The device recited in claim 7, wherein at least one said groove in each said pair of attachment devices comprises an open groove with a tubular cross-section, said open groove being adapted to have its open side forced apart to allow introduction of a flexible elongated member into said tubular cross-section, said open groove being adapted to close said tubular cross-section around said flexible elongated member and hold said flexible elongated member in place against transverse movement relative to said pad, but with insufficient friction to hold said flexible elongated member against axial movement within said tubular cross-section.

11. The device recited in claim 1, further comprising a label associated with each said pair of attachment devices, each said label being adapted to identify its associated said pair of said flexible elongated members.
